(19) United States
(12) Patent Application Publication
(10) Pub. No.: US 2022/0249598 A1
(43) Pub. Date: Aug. 11, 2022

(54) COMPOSITIONS TARGETING SPERM CALCINEURIN

(71) Applicant: Kent State University, Kent, OH (US)

(72) Inventors: Srinivasan VIJAYARAGHAVAN, Kent, OH (US); Souvik DEY, Kent, OH (US); Alaa EISA, Kent, OH (US)

(73) Assignee: Kent State University, Kent, OH (US)

(21) Appl. No.: 17/617,978

(22) PCT Filed: Jun. 10, 2020
(86) PCT No.: PCT/US20/36899

§ 371 Date: Dec. 10, 2021

Related U.S. Application Data
(60) Provisional application No. 62/859,225, filed on Jun. 10, 2019.

(51) Int. Cl. A61K 38/13; A61K 9/00; A61K 9/107; A61P 15/18; A61P 15/16; A61K 47/44; A61K 47/10; A61K 47/36

(52) U.S. Cl. CPC A61K 38/13; A61K 9/0034; A61K 9/107; A61K 47/36; A61P 15/16; A61K 47/44; A61K 47/10; A61P 15/18

(57) ABSTRACT

Compositions targeting sperm calcineurin may be used as topical and reversible contraceptives. The compositions may include calcineurin inhibitors such as tacrolimus (e.g. FK506) and/or Cyclosporin A. The compositions may be gels, creams, or lotion-based formulations and may be used reversibly and locally for both males and females.

This application claims the priority benefit of U.S. Provisional Application Ser. No. 62/859,225, filed Jun. 10, 2019, the contents of which are incorporated by reference herein.

### STATEMENT REGARDING FEDERALLY SPONSORED RESEARCH

This invention was made with government support under Grant/Contract No. 1R03HD096176-01 awarded by the National Institute of Health. The government has certain rights in the invention.

### BACKGROUND

The present disclosure relates to compositions that target sperm calcineurin. The compositions may be useful for contraceptives (e.g. male contraceptives) that are non-hormonal, reversible, inexpensive, and may be used topically. Methods of making and using (e.g. contraceptive methods) the compositions are also disclosed.

Presently, the global population is increasing at a magnitude of 80 million persons per year and is projected to surpass 9 billion within 2050. Furthermore, it is projected that almost half of all pregnancies globally are unplanned. Because of this rapid growth, unplanned gestation has severe mental and socio-economic consequences. The price of unintentional pregnancy in the United States only is assessed to be around 15 billion dollars. The rapid population growth and the astonishing figure of unplanned pregnancies are at least partially attributed to the deficiency of access to suitable contraception methods. The accessibility of abundant, useful and reversible contraceptive options for women has led to the female partner clearly assuming the primary responsibility for family planning. Although these choices have been available to females for the last 50 years, male options have remained inadequate.

Most known contraceptives are targeted towards females (mainly hormonal contraceptives with multiple profound side effects) and not towards males. Available male contraceptive methods either involve surgery (e.g. vasectomy) or injectables (e.g. Vasalgel), are sometimes not preferred by users (e.g. condoms) or have severe irreversible side effects (chemical-based contraceptives). Even the most recent male contraceptives are either hormone based or include polymers that may cause obstruction in the male reproductive tract. Therefore, they are likely to show side effects.

There is a need for a new reversible, user-friendly, topical contraceptive that is based on a non-hormonal formulation and could be used by both males and females.

### BRIEF DESCRIPTION

Treatment with calcineurin inhibitors, FK506 and Cyclosporin A, at nanomolar and micromolar levels, respectively have been found to significantly and independently inhibit the ability of the mouse sperm to fertilize eggs in vitro (i.e. outside body, in test tubes/culture plates). Contraceptives using FK506/cyclosporin A in gel/cream/lotion based formulations that could be used reversibly and locally without possible side effects both in males and females are disclosed. For females, the compositions could be used as an intra-vaginal gel and thus can inhibit the approaching sperm directly. For males, the compositions could be used as an ointment to be applied around scrotum that will penetrate into the testis and modulate the sperm. The developed non-hormonal contraceptive will be user-friendly and is likely to be inexpensive.

Disclosed, in some embodiments, is a non-hormonal contraceptive gel, cream, or lotion comprising at least one calcineurin inhibitor.

The at least one calcineurin inhibitor may be selected from the group consisting of Cyclosporin A and tacrolimus. For example, the calcineurin inhibitor(s) may include only one of Cyclosporin A or tacrolimus; or a combination thereof.

The at least one calcineurin inhibitor may present at a concentration of from about 1 to about 999 μM (i.e. a micromolar concentration).

Alternatively, the at least one calcineurin inhibitor may be present at a concentration of from about 1 to about 999 nM (i.e. a nanomolar concentration).

The lotion may be oil-based on water-based.

The gel may be oil-free.

Disclosed, in other embodiments, is a non-hormonal contraceptive gel composition comprising: water; at least one gelling agent; and at least one calcineurin inhibitor.

The at least one gelling agent may be selected from the group consisting of acacia, pectin, starch, tragacanth, xantham gum, alginic acid, cocoa butter, gelatin, bentonite, cellulose derivatives, carbomer resins, colloidal silicone dioxide, polyvinyl alcohol, mineral oil, and polyethylene gel.

Disclosed, in further embodiments, is a non-hormonal contraceptive cream composition comprising: an emulsion of water and oil; at least one calcineurin inhibitor.

The emulsion may be a water-in-oil emulsion or an oil-in-water emulsion.

Methods of making and using the contraceptive compositions of the present disclosure are also disclosed.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates the effect of treatments of mouse caudal spermatozoa with calcineurin and GSK3 inhibitors on in vitro fertilization, indicated by two-cell stage formation.

FIG. 2 illustrates the detection of FK506 induced defect in sperm motility and oocyte penetration.

FIG. 3 illustrates the regulation of GSK3α in mouse sperm by FK506.

FIG. 4 illustrates the elucidation of GSK3 and calcineurin interaction.

### DETAILED DESCRIPTION

The present disclosure may be understood more readily by reference to the following detailed description of desired embodiments included therein. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

FK506 binds with FKBP12 and this complex competitively inhibits calcineurin activity; as a result, calcineurin cannot dephosphorylate/activate GSK3-alpha. Reduced GSK3 activity hinders pyruvate uptake by the sperm cells. DRP, another substrate of calcineurin remains phosphorylated in the absence of calcineurin activity causing an anomaly in mitochondrial function. Ultimately, the inhibitor treated spermatozoa are mostly unable to exhibit hyperactivity during capacitation and thus are unable to penetrate and fertilize eggs in vitro.

Glycogen synthase kinase 3 (GSK3) was identified as an enzyme regulating sperm protein phosphatase. The isoform GSK3α, but not GSK3β, is essential for sperm function. Sperm lacking GSK3α display altered motility and an inability to undergo hyperactivation which is essential for fertilization. Male mice lacking calcineurin, a calcium regulated phosphatase, are also infertile. The loss of calcineurin results in impaired epididymal sperm maturation and motility. The phenotype of GSK3α and calcineurin knockout mice shared similarities prompting an examination of the interrelationship between the two enzymes in sperm. The fact that active calcineurin is required during their residence in the epididymis suggests that calcium levels are altered during epididymal sperm maturation. Total and free calcium levels are high in immotile compared to motile sperm. Inhibition of calcineurin by FK506 results in an increase in the net phosphorylation and a consequent decrease in catalytic activity of sperm GSK3. The inhibitor FK506 and an isoform specific inhibitor of GSK3α also inhibited fertilization of eggs in vitro. The interrelated functions of GSK3α and calcineurin are thus essential during epididymal sperm maturation and during fertilization. Male contraceptives targeting one or both of these enzymes should be effective.

Calcineurin inhibitors affect sperm production and motility parameters. The deletion of calcineurin results in male infertility in mice; the affected sperm display abnormal motility pattern with rigid midpiece that hampers their movements during hyperactivation. Injecting the male mice with FK506 also results with similar phenotypes for the sperm. A glycogen synthase kinase isoform (GSK3α, but not GSK3β), is essential for sperm function. Sperm lacking GSK3α display altered motility and an inability to undergo hyperactivation which is essential for fertilization; GSK3α knockout male mice are infertile. This similarity of the phenotypes in the sperm and fertility status of these two knockout lines led to an investigation of this interrelation using calcineurin inhibitors.

FK506 or tacrolimus and cyclosporin A are generally used as immunosuppressive drugs for allogenic organ transplantation to lower the risk of rejection. These drugs are also used to treat dry-eye syndrome and eczema. It has been demonstrated that tacrolimus inhibits T-lymphocyte activation by first binding to an intracellular protein, FKBP-12. A complex of tacrolimus-FKBP-12, calcium, calmodulin, and calcineurin is then formed and the phosphatase activity of calcineurin is inhibited. Bioavailability of FK506 from an ointment formulation is minimal (˜0.5%) and thus it does not pose any long term or systemic complications. However, 0.1% tacrolimus formulations cause increased sensitivity of skin, burning, stinging or itching sensations. This concentration roughly translates into nanomolar titer of the drug in blood, which can cause calcineurin inhibition of blood lymphocytes which may affect immunity phenotypes at genomic level. However, FK506 inhibits sperm calcineurin at nanomolar level, which when absorbed into the bloodstream could go down to as low as picomolar concentration. Therefore, the formulations of the present disclosure have advantages over the already available preparations used for other neural and immune diseases.

In some embodiments, the contraceptive composition is selected from creams, ointments, pastes, lotions, and gels. The contraceptive composition generally includes at least one calcineurin inhibitor at a micromolar or nanomolar concentration. In some embodiments, the concentration is in the range of about 10 nM to about 500 nM, including from about 15 nM to about 200 nM, and about 20 nM to about 100 nM. The at least one calcineurin inhibitor may be selected from Cyclosporin A, tacrolimus, and a mixture thereof. When Cyclosporin A and tacrolimus are included together, they may be present at approximately the same concentrations. Alternatively, Cyclosporin A may be at a higher concentration than tacrolimus or vice versa. Additional non-limiting examples of calcineurin inhibitors include fenvalerate ((RS)-alpha-Cyano-3-phenoxybenzyl (RS)-2-(4-chlorophenyl)-3-methylbutyrate), deltamethrin ([(S)-Cyano-(3-phenoxyphenyl)-methyl] (1R,3R)-3-(2,2-dibromoethenyl)-2,2-dimethyl-cyclopropane-1-carboxylate), 11R-CaN-AID, Calcineurin Autoinhibitory peptide ITSFEEAKGLDRINERMPPRRDAMP (aka Autoinhibitory peptide 457-482), and PP2B Inhibitor VIII.

The cream may be an emulsion of water and oil. The emulsion may be classified as an oil-in-water emulsion or a water-in-oil emulsion. In some embodiments, the cream is a vanishing cream or a cold cream.

The ointment may be a semi-solid preparation containing hydrocarbons (e.g. petrolatum, mineral oil, paraffins, synthetic hydrocarbons). In some embodiments, the ointment is devoid of water.

The paste may be a mixture of a powder with an ointment. The addition of the powder may enhance the breathability of the applied composition and/or enhance the consistency.

The lotion may be a liquid composition such as an oil-in-water emulsion with a sufficient water content to give the composition the liquid consistency. Optionally, the lotion may include a small amount of an alcohol.

The gel may contain one or more gelling agents in water or a water-alcohol mixture. The gel may be designed to liquify on contact with the skin. The at least one gelling agent may be selected from the group consisting of acacia, pectin, starch, tragacanth, xantham gum, alginic acid, cocoa butter, gelatin, bentonite, cellulose derivatives, carbomer resins, colloidal silicone dioxide, polyvinyl alcohol, mineral oil, polyethylene gel, and any mixture of two or more thereof.

Non-limiting examples of oils include mineral oil and various vegetable oils.

The contraceptive compositions of the present disclosure optionally include additives such as dispersants, viscosifiers, lubricants, antioxidants, tackifiers, wetting agents, dyes, pigments, fillers, fragrances, antibacterials, and/or essential oils.

Non-limiting examples of advantages of the contraceptive compositions and methods of the present disclosure include:
unlike many other alternatives, surgery is not required;
the formulation could be used locally, thereby reducing or eliminating the likelihood of any systemic side effects;
non-hormonal, thus not likely to affect other sexual parameters including libido;
low (e.g. micromolar or nanomolar) concentration, therefore more specific and less expensive compared available alternatives; and
not likely to affect male psychology, as is seen in case of use of condoms for some males.

The composition should be effective to modulate animal/human seminal sperm.

The gel, lotion, or cream formulation includes one or more calcineurin inhibitors, such as FK506, aka tacrolimus aka fujimycin and/or cyclosporin A.

The formulation is substantially effective with respect to the active principals (i.e. FK506/cyclosporin A).

The following examples are provided to illustrate the devices and methods of the present disclosure. The examples are merely illustrative and are not intended to limit the disclosure to the materials, conditions, or process parameters set forth therein.

### EXAMPLES

The importance of sperm calcineurin in fertilization of eggs was evaluated by using gene knockout as well as pharmacological inhibitor-based approaches. The knocking down of either of the calcineurin gene (ppp3r2 or ppp3cc) causes male infertility, and some biochemical parameters that can explain these observations were checked.

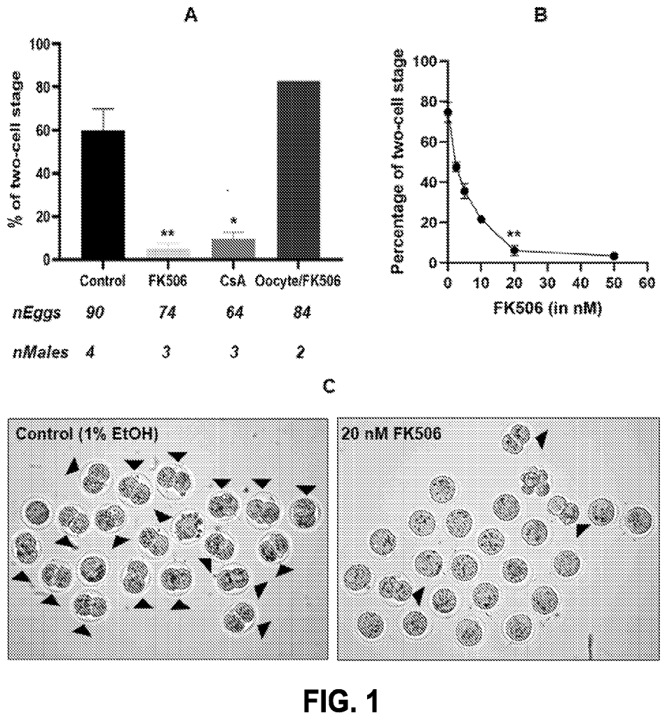

Inhibitor studies using FK506 and Cyclosporin A were conducted to check the necessity of calcineurin activity in sperm during capacitation and fertilization process (FIGS. 1-3). Nanomolar doses of FK506 were used to treat spermatozoa and then use those cells were used for in vitro fertilization (IVF) assays. The treated spermatozoa were exposed to three different types oocyte conditions while performing IVF: a) regular IVF with intact oocyte-cumulus complex, b) cumulus-free intact oocyte and c) cumulus-free zona-free oocyte.

Another set of treated sperm cell was analyzed for its motility parameters using computer assisted sperm analyzer (CASA). Average path velocity (VAP), curvilinear velocity (VCL) and amplitude of lateral head displacement (ALH) were measured to assess hyperactivation.

The effect of calcineurin inhibitor on GSK3αβ phosphorylation was studied by Western blot procedure. Calcium dependence of calcineurin mediated GSK3α regulation was also checked. Both GSK3 activity and Calcineurin activity were measured in presence of FK506.

Knockout of testis isoform-specific calcineurin regulatory subunit (ppp3r2-KO) were used to further validate the effect of loss of calcineurin on GSk3 phosphorylation and activity.

FIG. 1 illustrates the effect of treatments of mouse caudal spermatozoa with calcineurin and GSK3 inhibitors on in vitro fertilization, indicated by two-cell stage formation. A) Spermatozoa were incubated under capacitating medium (viz. Human tubal fluid/HTF or TYH medium) in presence of 1% ethanol (control), FK506 (20 nM), cyclosporin A (10 μM) for 1 hr at 37° C., inside 5% CO2 incubator. Additionally, oocytes were also incubated with same concentration of FK506, which it encounters during the IVF, the corresponding spermatozoa set was untreated. All data represent mean±SEM of n=3 for all the values. *p≤0.05 versus control. B) Dose response curve of FK506 at 0, 2.5, 5, 10, 20 & 50 nM concentration. C) Representative images showing effect of FK506 on IVF; black triangles directing the two-cell stage after fertilization of eggs.

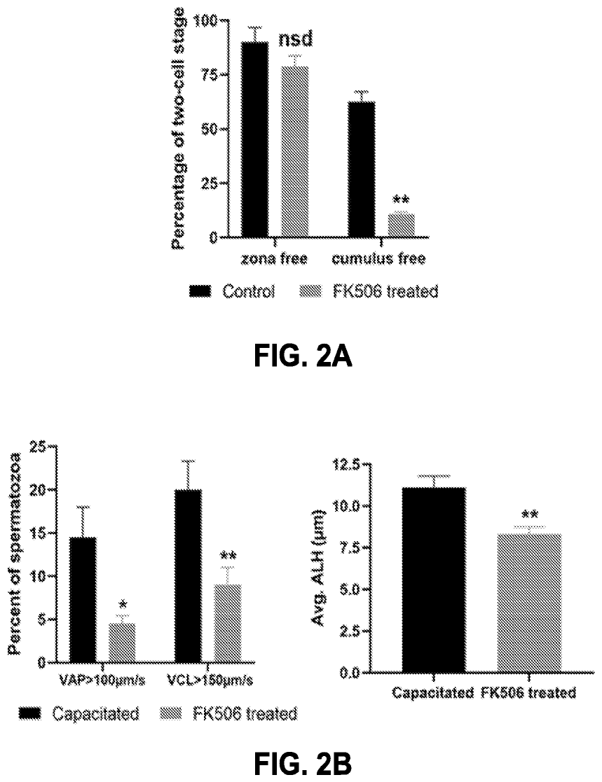

FIG. 2 illustrates the detection of FK506 induced defect in sperm motility and oocyte penetration. A) FK506 (20 nM) treated spermatozoa were used for both cumulus free zona intact oocytes as well as zona-free oocytes. B) Effect of FK506 treatment for (120-180 min) on sperm hyperactivation controlling parameters; VAP: average path velocity, VCL: curvilinear velocity, ALH: lateral head amplitude.

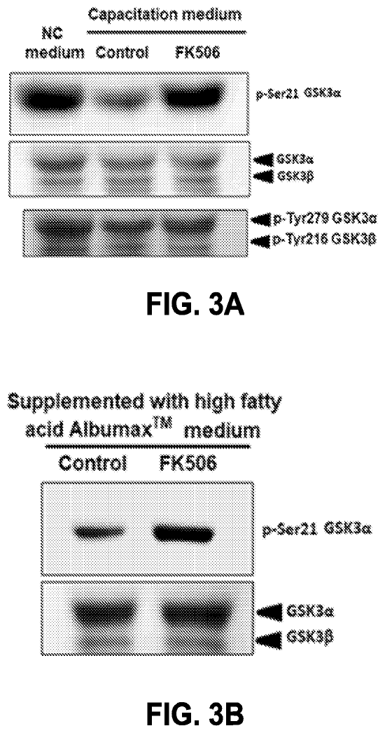

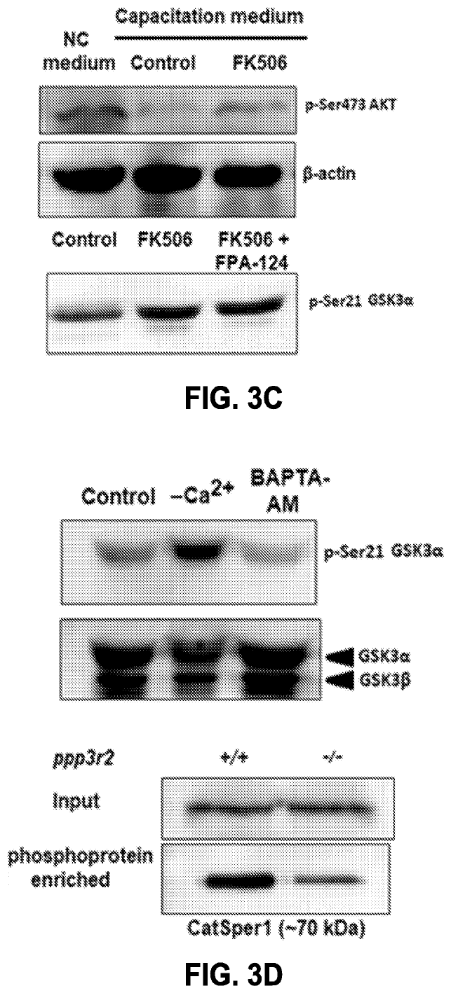

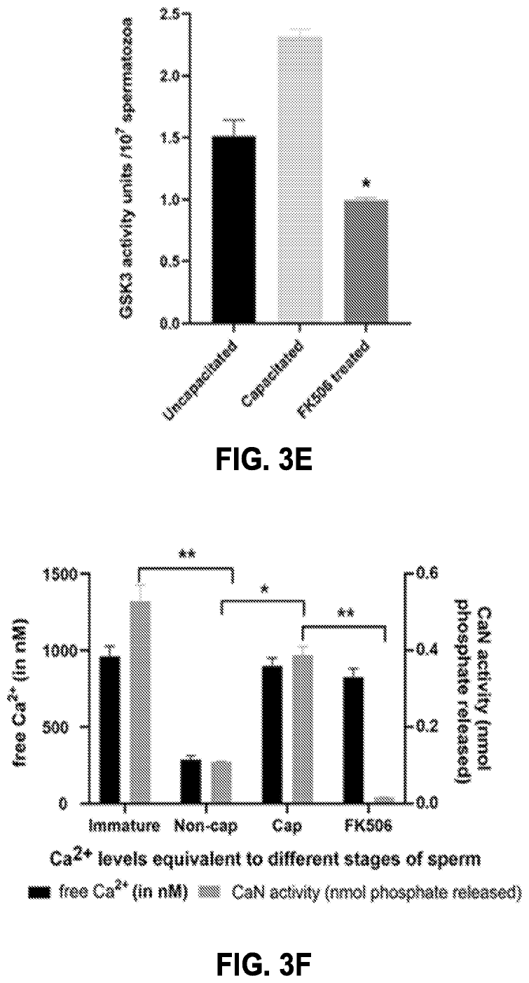

FIG. 3 illustrates the regulation of GSK3α in mouse sperm by FK506. A) Modulation of phospho-serine/tyrosine profile GSK3α by FK506 (20 nM) during capacitation; for capacitation spermatozoa were incubated with ‘capacitation medium’, HTF or TYH for ˜90 min. B) phosphor-serine GSK3α status was checked during capacitation with TYH medium having high fatty acid containing BSA. C) Possible involvement of AKT in mediation of calcineurin induced regulation of GSK3α. D) Validation of involvement of extracellular calcium in GSK3α regulation; no calcium salt was added in the ‘−Ca²⁺’ set, 10 μM BAPTA-AM was used to chelate intra-sperm Ca²⁺ ions; CatSper1 phosphorylation was compared between wild type and ppp3r2 knockout sperm extract E) GSK3 activity of sperm cells under capacitating condition was checked using p³²radioactive ATP for phosphorylation of substrate. F) Co-relation among intracellular free calcium levels of different maturational stages of sperm cells with calcineurin activity was measured; for negative control, 20 nM of FK506 was used. All data represent mean±SEM of n=4 for all the values. *p≤0.05, **p≤0.01 versus control.

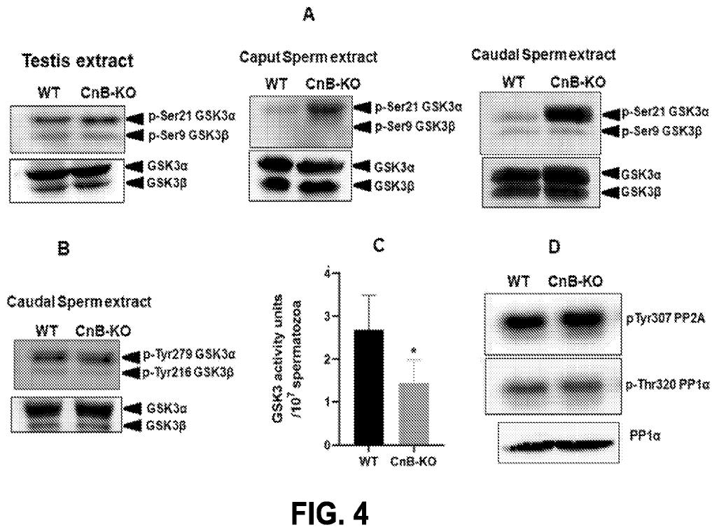

FIG. 4 illustrates the elucidation of GSK3 and calcineurin interaction. Profiling of GSK3 phosphorylation and associated activity in ppp3r2 knockout mice testis and sperm: A) GSK3 phospho-serine profiling and B) GSK3 phospho-tyrosine status of whole cell extract by Western blot. C) Comparison of GSK3 activity between wild type and ppp3r2 knockout mice caudal sperm extract; data represent mean±SEM of n=4 for all the values. *p≤0.05 versus wild type. D) phosphorylation status of PP2A and PP1α (pp1y2) in ppp3r2 knockout mice caudal sperm extract.

Some of the key observations include:

Both the calcineurin inhibitors, FK506 and cyclosporin A were able to significantly inhibit the fertilizing ability of sperm in vitro (FIG. 1A).

Pre-treatment of oocytes with the same concentration of FK506 did not result in any reduction in fertilization, indicates that oocytes were unaffected by the inhibitor (FIG. 1B).

Nanomolar doses of FK506 were good enough to inhibit sperm cells in their ability to fertilize eggs in vitro (FIG. 1C).

The inhibition was not affecting sperm cells to fertilize zona-free eggs (FIG. 2A) and it was due to lack of attainment of hyperactivation of the sperm under influence of the inhibitor (FIG. 2B).

Calcineurin affects GSK3α phosphorylation as well as activity and this is dependent upon intake of external calcium ion by the sperm cells (FIG. 3A-F).

Deletion of calcineurin (ppp3r2) gene results in reduced GSK3 activity, confirming the observations recorded using Fk506 study.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A non-hormonal contraceptive gel, cream, or lotion comprising at least one calcineurin inhibitor.

2. The non-hormonal contraceptive gel, cream, or lotion of claim 1, wherein the at least one calcineurin inhibitor is selected from the group consisting of Cyclosporin A and tacrolimus.

3. The non-hormonal contraceptive gel, cream, or lotion of claim 1, wherein the at least one calcineurin inhibitor consists of Cyclosporin A.

4. The non-hormonal contraceptive gel, cream, or lotion of claim 1, wherein the at least one calcineurin inhibitor consists of tacrolimus.

5. The non-hormonal contraceptive gel, cream, or lotion of claim 1, wherein the at least one calcineurin inhibitor comprises Cyclosporin A and tacrolimus.

6. The non-hormonal contraceptive gel, cream, or lotion of claim 1, wherein the at least one calcineurin inhibitor is present at a concentration of from about 1 to about 999 μM.

7. The non-hormonal contraceptive gel, cream, or lotion of claim 1, wherein the at least one calcineurin inhibitor is present at a concentration of from about 1 to about 999 nM.

8. The non-hormonal contraceptive gel, cream, or lotion of claim 1, wherein the gel, cream, or lotion is a cream.

9. The non-hormonal contraceptive gel, cream, or lotion of claim 1, wherein the gel, cream, or lotion is an oil-based lotion.

10. The non-hormonal contraceptive gel, cream, or lotion of claim 1, wherein the gel, cream, or lotion is water-based lotion.

11. The non-hormonal contraceptive gel, cream, or lotion of claim 1, wherein the gel, cream, or lotion is an oil-free gel.

12. The non-hormonal contraceptive gel, cream, or lotion of claim 1, wherein the gel, cream, or lotion is water-based.

13. The non-hormonal contraceptive gel, cream, or lotion of claim 1, wherein the gel, cream, or lotion is oil-based.

14. A non-hormonal contraceptive gel composition comprising:
water;
at least one gelling agent; and
at least one calcineurin inhibitor.

15. The non-hormonal contraceptive gel composition of claim 14, wherein the at least one calcineurin inhibitor is selected from the group consisting of Cyclosporin A and tacrolimus.

16. The non-hormonal contraceptive gel composition of claim 14, wherein the at least one gelling agent is selected from the group consisting of acacia, pectin, starch, tragacanth, xantham gum, alginic acid, cocoa butter, gelatin, bentonite, cellulose derivatives, carbomer resins, colloidal silicone dioxide, polyvinyl alcohol, mineral oil, and polyethylene gel.

17. A non-hormonal contraceptive cream composition comprising:
an emulsion of water and oil;
at least one calcineurin inhibitor.

18. The non-hormonal contraceptive cream composition of claim 17, wherein the emulsion is a water-in-oil emulsion.

19. The non-hormonal contraceptive cream composition of claim 17, wherein the emulsion is an oil-in-water emulsion.
